(19) United States
(12) Patent Application Publication
(10) Pub. No.: US 2021/0060288 A1
(43) Pub. Date: Mar. 4, 2021

(54) BREAST-COMFORTING DEVICE

(71) Applicant: Briana Julius, Lebanon, IL (US)

(72) Inventor: Briana Julius, Lebanon, IL (US)

(21) Appl. No.: 16/552,936

(22) Filed: Aug. 27, 2019

(51) Int. Cl. A61M 21/02; A61H 23/00; A61F 7/02

(52) U.S. Cl. CPC A61M 21/02; A61F 2007/0021; A61F 7/02; A61H 23/00

(57) ABSTRACT

A breast-comforting device, including a plurality of comforting members, including a left pad to receive at least a portion of a first breast of a user, the left pad including a heating unit disposed within at least a portion of the left pad to disperse heat on at least a portion of an inner surface of the left pad, and a pressure dissemination unit disposed within at least a portion of the left pad to provide a pressured sensation to at least a portion of the first breast, and a right pad to receive at least a portion of a second breast of the user, the right pad including another heating unit disposed within at least a portion of the right pad to disperse heat on at least a portion of an inner surface of the right pad, and another pressure dissemination unit disposed within at least a portion of the right pad to provide a pressured sensation to at least a portion of the second breast, and an input unit connected to the plurality of comforting members to manipulate at least one of the plurality of comforting members.

### BACKGROUND

### 1. Field

The present general inventive concept relates generally to a comforting device, and particularly, to a breast-comforting device.

### 2. Description of the Related Art

At the start of the twentieth century, breast-feeding developed a negative reputation as an activity that only poor, working-class mothers would engage in, and the bottle-feeding of babies with baby formula became the norm. However, in the 1960's, scientists discovered that breast-feeding actually provides babies with considerable psychological and physiological benefits, and breast-feeding has been increasing in popularity ever since.

Unfortunately, breast-feeding does tend to create some appreciable physiological problems for the breast-feeding mother. Most breast-feeding mothers start experiencing breast pain, the formation of lumps and knots within the breast, and the development of engorgement and clogged milk ducts. For over a hundred years, the only solution that medical science could suggest for these problems was for the mother to manually massage her own breast or warm the breasts up by means of a heating pad or a hot shower.

Therefore, there is a need for a breast-comforting device that can relieve the pain associated with breast-feeding.

### SUMMARY

The present general inventive concept provides a breast-comforting device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a breast-comforting device, including a plurality of comforting members, including a left pad to receive at least a portion of a first breast of a user, the left pad including a heating unit disposed within at least a portion of the left pad to disperse heat on at least a portion of an inner surface of the left pad, and a pressure dissemination unit disposed within at least a portion of the left pad to provide a pressured sensation to at least a portion of the first breast, and a right pad to receive at least a portion of a second breast of the user, the right pad including another heating unit disposed within at least a portion of the right pad to disperse heat on at least a portion of an inner surface of the right pad, and another pressure dissemination unit disposed within at least a portion of the right pad to provide a pressured sensation to at least a portion of the second breast, and an input unit connected to the plurality of comforting members to manipulate at least one of the plurality of comforting members.

The input unit may include a controller to control at least one of a heat level, a vibration speed, and a vibration intensity.

The breast-comforting device may further include a plurality of aromatic units disposed within at least a portion of the plurality of comforting members to dispense at least one of a fragrance and a massage oil to facilitate relaxation.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a top perspective view of a breast-comforting device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the FIGURES, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Breast-Comforting Device 100
Comforting Members 110
Left Pad 111
Outer Surface 111a
Inner Surface 111b
Pump-Receiving Aperture 111c
Fastener 111d
Heating Unit 111e
Pressure Dissemination Unit 111f
Right Pad 112
Outer Surface 112a
Inner Surface 112b
Pump-Receiving Aperture 112c
Fastener 112d
Heating Unit 112e
Pressure Dissemination Unit 112f
Torso Strap 113
Input Unit 120
Controller 121
Left Pad Selector 121a
Right Pad Selector 121b
Dual Pad Selector 121c
Pressure Dissemination Selector 121d
Heating Selector 121e
Power Selector 121f
Slider 121g
Cord 122
Aromatic Units 130

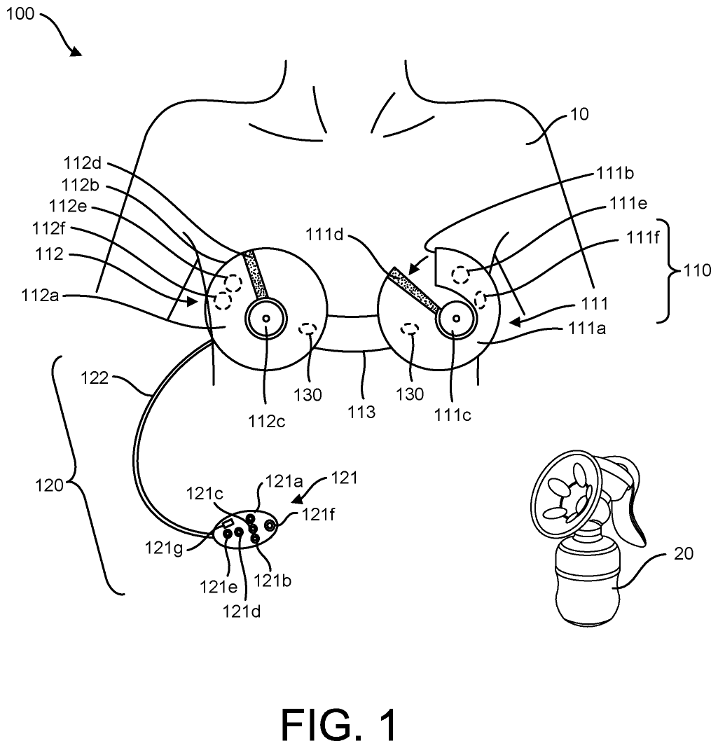

FIG. 1 illustrates a top perspective view of a breast-comforting device 100, according to an exemplary embodiment of the present general inventive concept.

The breast-comforting device 100 may be constructed from at least one of metal, plastic, wood, glass, and rubber, etc., but is not limited thereto.

The breast-comforting device 100 may include a plurality of comforting members 110, an input unit 120, and a plurality of aromatic units 130, but is not limited thereto.

The plurality of comforting members 110 may include a left pad 111, a right pad 112, and a torso strap 113, but is not limited thereto.

The left pad 111 may include an outer surface 111a, an inner surface 111b, a pump-receiving aperture 111c, a fastener 111d, a heating unit 111e, and a pressure dissemination unit 111f, but is not limited thereto.

The left pad 111 may be of any predetermined size to fit on and/or around at least a portion of a first breast of a user 10. The outer surface 111a may be a smooth surface and/or a textured surface to facilitate gripping thereof, such as a plurality of bubbled protrusions extending away therefrom. The inner surface 111b may be another smooth surface and/or a cushion to enhance comfort while disposed on and/or around at least a portion of the first breast of the user 10. In other words, the left pad 111 may receive at least a portion of the first breast therein.

Additionally, the left pad 111 may be adjustable to accommodate a size and circumference of the first breast of the user 10.

The pump-receiving aperture 111c may form at a center portion of the left pad 111 in response to wrapping the left pad 111 around the first breast of the user 10. As such, the pump-receiving aperture 111c may at least partially expose a portion of a nipple and/or an areola of the first breast of the user 10. As such, the pump-receiving aperture 111c may facilitate connecting a breast pump 20 to the first breast of the user 10.

The fastener 111d may include hooks and loops, a button, a clasp, a clamp, and a snap, but is not limited thereto.

The fastener 111d may be disposed on at least a portion of the left pad 111. The fastener 111d may be used to adjust and/or tighten the left pad 111 on and/or around the first breast of the user 10, such that the left pad 111 may be prevented from falling off the first breast of the user 10.

The heating unit 111e may include an electrical coil, but is not limited thereto.

The heating unit 111e may disperse heat within at least a portion of the left pad 111. Specifically, the heating unit 111e may disperse heat on at least a portion of the inner surface 111b of the left pad 111, such that the first breast of the user 10 may receive heat thereupon. As such, the first breast of the user 10 may feel relief from discomfort during and/or after breast-feeding. Furthermore, the left pad 111 may relieve discomfort from at least one of engorgement, lumps, knots, and clogged milk ducts.

The pressure dissemination unit 111f may include various devices to simulate a massage, including, but not limited to vibration devices, pistons, mechanical rollers, compression devices, stretching devices, kneading devices, pressing devices, oscillators, gears, etc. As such, the left pad 111 may provide a pressured sensation to the first breast of the user 10 in response to the user 10 wearing the left pad 111, and activating the pressure dissemination unit 111f, thus simulating a massage.

As such, the first breast of the user 10 may feel relief from discomfort during and/or after breast-feeding. Furthermore, the left pad 111 may relieve discomfort from at least one of engorgement, lumps, knots, and clogged milk ducts.

The pressure dissemination unit 111f may be of various shapes including, but not limited to, ball-shaped, round, oval, circular, rectangular, prism, rod-shaped, and/or conical. The pressure dissemination unit 111f may minimize discomfort to the user 10 depending on placement therethroughout the left pad 111.

The right pad 112 may include an outer surface 112a, an inner surface 112b, a pump-receiving aperture 112c, a fastener 112d, a heating unit 112e, and a pressure dissemination unit 112f, but is not limited thereto.

The right pad 112 may be of any predetermined size to fit on and/or around at least a portion of a second breast of the user 10. The outer surface 112a may be a smooth surface and/or a textured surface to facilitate gripping thereof, such as a plurality of bubbled protrusions extending away therefrom. The inner surface 112b may be another smooth surface and/or a cushion to enhance comfort in response to being disposed on and/or around at least a portion of the second breast of the user 10. In other words, the right pad 112 may receive at least a portion of the second breast therein.

Additionally, the right pad 112 may be adjustable to accommodate a size and circumference of the second breast of the user 10.

The pump-receiving aperture 112c may form at a center portion of the right pad 112 in response to wrapping the right pad 112 around the second breast of the user 10. As such, the pump-receiving aperture 112c may at least partially expose a portion of a nipple and/or an areola of the second breast of the user 10. As such, the pump-receiving aperture 112c may facilitate connecting the breast pump 20 to the second breast of the user 10.

The fastener 112d may include hooks and loops, a button, a clasp, a clamp, and a snap, but is not limited thereto.

The fastener 112d may be disposed on at least a portion of the right pad 112. The fastener 112d may be used to adjust and/or tighten the right pad 112 on and/or around the second breast of the user 10, such that the right pad 112 may be prevented from falling off the second breast of the user 10.

The heating unit 112e may include an electrical coil, but is not limited thereto.

The heating unit 112e may disperse heat within at least a portion of the right pad 112. Specifically, the heating unit 112e may disperse heat on at least a portion of the inner surface 111b of the right pad 112, such that the second breast of the user 10 may receive heat thereupon. As such, the second breast of the user 10 may feel relief from discomfort during and/or after breast-feeding. Furthermore, the right pad 112 may relieve discomfort from at least one of engorgement, lumps, knots, and clogged milk ducts.

The pressure dissemination unit 112f may include various devices to simulate a massage, including, but not limited to, vibration devices, pistons, mechanical rollers, compression devices, stretching devices, kneading devices, pressing devices, oscillators, gears, etc. As such, the right pad 112 may provide a pressured sensation to the second breast of the user 10 in response to the user 10 wearing the right pad 112, and activating the pressure dissemination unit 112f, thus simulating a massage.

As such, the second breast of the user 10 may feel relief from discomfort during and/or after breast-feeding. Furthermore, the right pad 112 may relieve discomfort from at least one of engorgement, lumps, knots, and clogged milk ducts.

The pressure dissemination unit 112f may be of various shapes including, but not limited to, ball-shaped, round, oval, circular, rectangular, prism, rod-shaped, and/or conical. The pressure dissemination unit 112f may minimize discomfort to the user 10 depending on placement therethroughout the right pad 112.

The torso strap 113 may be disposed between the left pad 111 and the right pad 112. Moreover, the torso strap 113 may be disposed on at least a portion of the left pad 111 and/or at least a portion of the right pad 112.

The torso strap 113 may be removably disposed on at least a portion of a torso of the user 10, such that the torso strap 113 may support the left pad 111 and/or the right pad 112 to prevent the left pad 111 and/or the right pad 112 from falling off the first breast and/or the second breast of the user 10. As such, the torso strap 113 may be wrapped around at least a portion of the torso of the user 10.

The input unit 120 may include a controller 121 and a cord 122, but is not limited thereto.

The controller 121 may include a left pad selector 121a, a right pad selector 121b, a dual pad selector 121c, a pressure dissemination selector 121d, a heating selector 121e, a power selector 121f, and a slider 121g, but is not limited thereto.

The controller 121 may manipulate at least one of the left pad 111 and the right pad 112.

The left pad selector 121a may include a button, a switch, a lever, and a dial, but is not limited thereto.

The left pad selector 121a may be depressed to select the left pad 111.

The right pad selector 121b may include a button, a switch, a lever, and a dial, but is not limited thereto.

The right pad selector 121b may be depressed to select the right pad 112.

The dual pad selector 121c may include a button, a switch, a lever, and a dial, but is not limited thereto.

The dual pad selector 121c may be depressed to select the left pad 111 and the right pad 112.

The pressure dissemination selector 121d may include a button, a switch, a lever, and a dial, but is not limited thereto.

The pressure dissemination selector 121d may activate and/or deactivate the pressure dissemination unit 111f and/or the pressure dissemination unit 112f based on whether the left pad selector 121a, the right pad selector 121b, or the dual pad selector 121c was depressed. The pressure dissemination selector 121d may be pressed a predetermined number of times and/or a predetermined duration of time to adjust a pre-programmed massage style, such as Swedish, deep tissue, myofacial release, shiatsu, Thai, simulation of fingers, elbows, hands chopping, vibration, reiki, reflexology, and/or pounding.

The heating selector 121e may include a button, a switch, a lever, and a dial, but is not limited thereto.

The heating selector may activate and/or deactivate the heating unit 111e and/or the heating unit 112e based on whether the left pad selector 121a, the right pad selector 121b, or the dual pad selector 121c was depressed.

The slider 121g may be moved in a first direction to increase a vibration speed and/or a vibration intensity of the pressure dissemination unit 111f and/or the pressure dissemination unit 112f, based on whether the left pad selector 121a, the right pad selector 121b, or the dual pad selector 121c was depressed. Alternatively, the slider 121g may be moved in a second direction to decrease the vibration speed and/or the vibration intensity of the pressure dissemination unit 111f and/or the pressure dissemination unit 112f, based on whether the left pad selector 121a, the right pad selector 121b, or the dual pad selector 121c was depressed.

Also, the slider 121g may be moved in the first direction to increase a heat level of the heating unit 111e and/or the heating unit 112e, based on whether the left pad selector 121a, the right pad selector 121b, and the dual pad selector 121c was depressed. Alternatively, the slider 121g may be moved in the second direction to decrease the heat level of the heating unit 111e and/or the heating unit 112e, based on whether the left pad selector 121a, the right pad selector 121b, and the dual pad selector 121c was depressed.

Furthermore, the heating unit 111e, the pressure dissemination unit 111f, the heating unit 112e, and/or the pressure dissemination unit 112f may be independently controlled via the controller 121. For example, the user 10 may activate the pressure dissemination unit 111f of the left pad 111 to apply a deep tissue massage, while the user 10 activates the heating unit 112f of the right pad 112 to apply heat only.

The cord 122 connects the controller 121 to the plurality of comforting members 110.

Referring to FIG. 1, the cord 122 is illustrated as a wired connection between the controller 121 and the plurality of comforting members 110. However, communication between the controller 121 and the plurality of comforting members 110 may occur with a wireless connection using BLUETOOTH, near-field communication (NFC), WIFI, satellite, or RFID communication, but is not limited thereto. For example, the plurality of comforting members 110 may further include a communication unit (not illustrated) disposed within at least a portion thereof.

The plurality of aromatic units 130 may include a fragrance dispenser and a liquid dispenser, but is not limited thereto.

The plurality of aromatic units 130 may be disposed within at least a portion of the left pad 111 and the right pad 112, but is not limited thereto. The plurality of aromatic units 130 may dispense a fragrance in response to an activation of at least of the left pad 111 and the right pad 112. As such, the plurality of aromatic units 130 may provide an aromatherapy experience.

Moreover, the plurality of aromatic units 130 may dispense a liquid in response to another activation of at least one of the left pad 111 and the right pad 112, such as a massage oil and/or a relaxation oil on at least a portion of the left pad 111 and/or the right pad 112, such that the massage oil and/or the relaxation oil facilitates relaxation of the first breast and/or the second breast by the user 10.

As such, the heating unit 111e, the pressure dissemination unit 111f, the heating unit 112e, and/or the pressure dissemination unit 112f may simulate a hot stone massage and/or a warm oil massage, for added therapeutic value.

The present general inventive concept may include a breast-comforting device 100, including a plurality of comforting members 110, including a left pad 111 to receive at least a portion of a first breast of a user 10, the left pad 111 including a heating unit 111e disposed within at least a portion of the left pad 111 to disperse heat on at least a portion of an inner surface 111b of the left pad 111, and a pressure dissemination unit 111f disposed within at least a portion of the left pad 111 to provide a pressured sensation to at least a portion of the first breast, and a right pad 112 to receive at least a portion of a second breast of the user 10, the right pad 112 including another heating unit 112e disposed within at least a portion of the right pad 112 to disperse heat on at least a portion of an inner surface 112b of the right pad 112, and another pressure dissemination unit 112f disposed within at least a portion of the right pad 112 to provide a pressured sensation to at least a portion of the second breast, and an input unit 120 connected to the plurality of comforting members 110 to manipulate at least one of the plurality of comforting members 110.

The input unit 120 may include a controller 121 to control at least one of a heat level, a vibration speed, and a vibration intensity.

The breast-comforting device 100 may further include a plurality of aromatic units 130 disposed within at least a portion of the plurality of comforting members 110 to dispense at least one of a fragrance and a massage oil to facilitate relaxation.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A breast-comforting device, comprising:
a plurality of comforting members, comprising:
a left pad to receive at least a portion of a first breast of a user, the left pad comprising:
a heating unit disposed within at least a portion of the left pad to disperse heat on at least a portion of an inner surface of the left pad, and
a pressure dissemination unit disposed within at least a portion of the left pad to provide a pressured sensation to at least a portion of the first breast, and
a right pad to receive at least a portion of a second breast of the user, the right pad comprising:
another heating unit disposed within at least a portion of the right pad to disperse heat on at least a portion of an inner surface of the right pad, and
another pressure dissemination unit disposed within at least a portion of the right pad to provide a pressured sensation to at least a portion of the second breast; and
an input unit connected to the plurality of comforting members to manipulate at least one of the plurality of comforting members.

2. The breast-comforting device of claim 1, wherein the input unit comprises:
a controller to control at least one of a heat level, a vibration speed, and a vibration intensity.

3. The breast-comforting device of claim 1, further comprising:
a plurality of aromatic units disposed within at least a portion of the plurality of comforting members to dispense at least one of a fragrance and a massage oil to facilitate relaxation.
